(19) United States
(12) Patent Application Publication
(10) Pub. No.: US 2023/0398526 A1
(43) Pub. Date: Dec. 14, 2023

(54) NITROGEN HETEROCYCLIC CARBENE CATALYST AND PREPARATION METHOD THEREFOR

(71) Applicant: SHENZHEN BAY LABORATORY PINGSHAN TRANSLATIONAL MEDICINE CENTER, Guangdong (CN)

(72) Inventors: Jie'an CHEN, Shenzhen, Guangdong Province (CN); Yong HUANG, Shenzhen, Guangdong Province (CN); Fangfang GUO, Shenzhen, Guangdong Province (CN)

(21) Appl. No.: 18/037,621

(22) PCT Filed: Nov. 18, 2020
(86) PCT No.: PCT/CN2020/129804

§ 371 Date: May 18, 2023

(51) Int. Cl. B01J 31/02; B01J 37/16

(52) U.S. Cl. CPC B01J 31/0247; B01J 37/16

(57) ABSTRACT

A nitrogen heterocyclic carbene catalyst, having a structural formula represented by formula I:

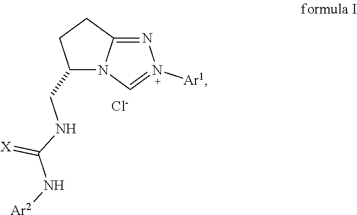

Ar¹ and Ar² in the formula I represent any one independently selected from the group consisting of an aryl, a substituted aryl, a heteroaryl, and a substituted heteroaryl, respectively; and X represents an oxygen atom or a sulfur atom. Thiourea (urea) is introduced to the nitrogen heterocyclic carbene skeleton as another active site, which can form various bifunctional nitrogen heterocyclic carbene catalysts.

### TECHNICAL FIELD

The present application relates to the technical field of catalytic materials, and more particularly to a nitrogen heterocyclic carbene catalyst and a preparation method therefor.

### BACKGROUND

In recent years, catalysts having dual functional groups and dual catalytic sites have been developed and successfully applied in organic asymmetric catalysis. A chiral catalyst containing thiourea (urea) group has become a most widely used bifunctional catalyst, due that thiourea groups in its structure form hydrogen bonds with a variety of substrates to achieve good catalytic activity and stereoselectivity. For example, a quinine thiourea catalyst, a chiral cyclohexanediamine thiourea catalyst, a phosphonimide thiourea catalyst, and the like. Bifunctional thiourea catalysts have been widely used in asymmetric aza-Michael reactions.

As an important class of organic small molecule catalysts, nitrogen heterocyclic carbenes (N-Heterocyclic Carbenes, NHCs) have been widely used in the field of organic asymmetric catalysis. However, the currently developed bifunctional nitrogen heterocyclic carbene catalysts have not been successfully applied to the asymmetric aza-Michael addition reactions.

### Technical Problems

It is one of objectives of embodiments of the present application to provide a nitrogen heterocyclic carbene catalyst and a preparation method therefor, so as to solve the technical problem that the existing bifunctional nitrogen heterocyclic carbenes can only catalyze limited reaction types.

### Technical Solutions

In order to solve the above technical problems, the present application adopts the following technical solutions:

In a first aspect, a nitrogen heterocyclic carbene catalyst is provided. The nitrogen heterocyclic carbene catalyst has a structural formula represented by formula I:

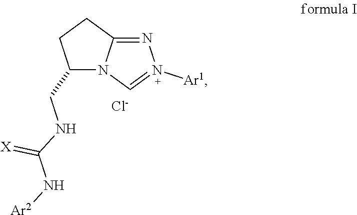

in which, Ar₁ and Ar₂ in the formula I represent any one independently selected from the group consisting of an aryl, a substituted aryl, a heteroaryl, and a substituted heteroaryl, respectively; and X represents an oxygen atom or a sulfur atom.

In a second aspect, a method for preparing a nitrogen heterocyclic carbene catalyst is provided. The method comprises steps of:

enabling (S)-2-(azidomethyl)-5-methoxy-3,4-dihydro-2H-pyrrole represented by formula V to react with Ar₁NHNH₂·HCl to obtain a compound represented by formula VI;

enabling the compound represented by formula VI to react with trimethyl orthoformate and hydrochloric acid to obtain a compound represented by formula VII;

subjecting the compound represented by formula VII to a hydrogenation reduction to obtain a compound represented by formula VIII; and

enabling the compound represented by formula VIII to react with an isothiocyanate Ar₂NCX to obtain the nitrogen heterocyclic carbene catalyst represented by formula I.

Structural formulas of above compounds are as follows:

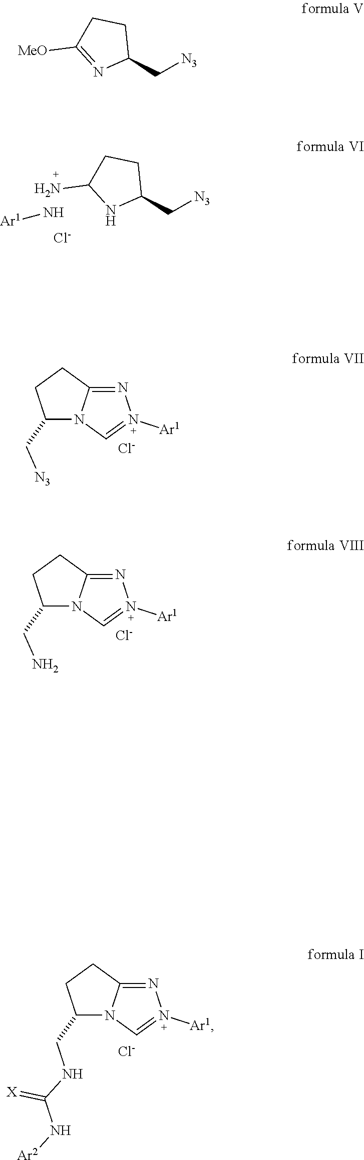

in which, Ar₁ and Ar₂ in the formula I represent any one independently selected from the group consisting of an aryl, a substituted aryl, a heteroaryl, and a substituted heteroaryl, respectively; and X represents an oxygen atom or a sulfur atom.

### BENEFICIAL EFFECTS

Advantages of the nitrogen heterocyclic carbene catalyst provided by embodiments of the present application are summarized as follows: in the present application, thiourea (urea) is introduced to the nitrogen heterocyclic carbene skeleton as another active site, and Ar₁ and Ar₂ in formula I are respectively selected from multiple groups including aryls, substituted aryls, heteroaryls, and substituted heteroaryls, so as to form the bifunctional thiourea catalyst, that is, the nitrogen heterocyclic carbene catalyst. The nitrogen heterocyclic carbine catalyst has good catalytic activity in catalyzing asymmetric aza-Michael reactions, thereby having a potential application in the field of organic asymmetric catalysis.

Advantages of the method for preparing the nitrogen heterocyclic carbene catalyst provided by embodiments of the present application are summarized as follows: the preparation method is advantageous in easily accessible raw materials, low cost, convenient operation, mild reaction conditions, and easy control, and can be prepared into the bifunctional thiourea catalyst, that is, the nitrogen heterocyclic carbene catalyst, which has good catalytic activity in catalyzing asymmetric aza-Michael reactions, thereby having a potential application in the field of organic asymmetric catalysis.

### BRIEF DESCRIPTION OF THE DRAWINGS

In order to more clearly illustrate the technical solutions in the embodiments of the present application, the drawings that need to be used in the description of the embodiments or the prior art will be briefly described hereinbelow. Obviously, the accompanying drawings in the following description are only some embodiments of the present application. For those skilled in the art, other drawings can be obtained based on these drawings without creative work.

FIG. 1 is a schematic flow chart of a method for preparing a nitrogen heterocyclic carbene catalyst according to an embodiment of the present application.

### DETAILED DESCRIPTION OF THE EMBODIMENTS

In order to make the purposes, technical solutions, and advantages of the present application clearer and more understandable, the present application will be further described in detail hereinafter with reference to the accompanying drawings and embodiments. It should be understood that the embodiments described herein are only intended to illustrate but not to limit the present application.

Compounds and derivatives thereof involved in embodiments/examples of the present application are all named according to the nomenclature system of International Union of Pure and Applied Chemistry (IUPAC) or Chemical Abstracts Service (CAS). Therefore, the groups of compounds specifically involved in the embodiments/example of the present application are described and illustrated as follows:

“Aryl” refers to an aromatic hydrocarbon having an aromatic ring structure, including but not limited to phenyl, naphthyl, anthracenyl, phenanthrenyl, and other similar groups. “Substituted aryl” refers to aryl derivatives in which, at least one hydrogen atom on the aryl ring is replaced by other functional groups, in which the substituted functional groups can be one or more.

“Heteroaryl” refers to a monocyclic or polycyclic or fused-ring aromatic hydrocarbon in which one or more carbon atoms have been replaced by a heteroatom such as nitrogen, oxygen, or sulfur. If a heteroaryl contains more than one heteroatom, the heteroatoms may or may not be the same. The heteroaryls include, but are not limited to, benzofuryl, benzothienyl, benzimidazolyl, thiazolyl, thienyl, and other similar groups. “Substituted heteroaryl” refers to heteroaryl derivatives in which, at least one of the hydrogen atoms on the above-mentioned heteroaryl ring is replaced by other functional groups, in which, the substituted functional groups may be one or more.

In an aspect, the present application provides a nitrogen heterocyclic carbene catalyst, having a structural formula represented by formula I:

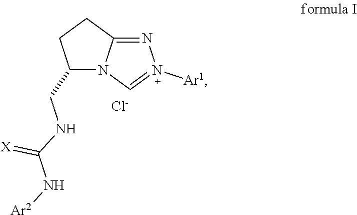

in which, Ar₁ and Ar₂ in the formula I represent any one independently selected from the group consisting of an aryl, a substituted aryl, a heteroaryl, and a substituted heteroaryl, respectively; and X represents an oxygen atom or a sulfur atom.

In the nitrogen heterocyclic carbene catalyst provided by embodiments of the present application, thiourea (urea) is introduced to the nitrogen heterocyclic carbene skeleton as another active site, and Ar₁ and Ar₂ in formula I are respectively selected from multiple groups including aryls, substituted aryls, heteroaryls, and substituted heteroaryls, so as to form the bifunctional thiourea catalyst, that is, the nitrogen heterocyclic carbene catalyst. The nitrogen heterocyclic carbine catalyst has good catalytic activity in catalyzing asymmetric aza-Michael reactions, thereby having a potential application in the field of organic asymmetric catalysis.

In some embodiments, An and Are can be the same or different aryls, substituted aryls, heteroaryls, and substituted heteroaryls.

Specifically, the aryl group is at least one selected from the group consisting of phenyl, naphthyl, anthracenyl, phenanthrenyl, and fluorenyl. The substituted aryl refers to the above aryls introduced with substituents. Specifically, the substituted aryl is at least one selected from the group consisting of a substituted phenyl, a substituted naphthyl, a substituted anthracenyl, a substituted phenanthrenyl, and a substituted fluorenyl. A substituent in the substituted aryl is at least one selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

The heteroaryl is at least one selected from the group consisting of a monocyclic heteroaryl and a fused-ring heteroaryl. The monocyclic heteroaryl is at least one selected from the group consisting of furyl, thienyl, pyrrolyl, imidazolyl, pyrazolyl, oxazolyl, thiazolyl, pyridinyl, pyranyl, pyrimidinyl, and pyrazinyl. The fused-ring heteroaryl is at least one selected from the group consisting of benzofuryl, benzothienyl, benzopyrrolyl, benzimidazolyl, benzoxazolyl, benzopyrazolyl, benzothiazolyl, benzopyranyl, quinolone, and acridine. The substituted heteroaryl refers to the above heteroaryls introduced with substituents. The substituted heteroaryl is selected from at least one of a substituted monocyclic heteroaryl and a substituted fused-ring heteroaryl. A substituted monocyclic heteroaryl is selected from the group consisting of a substituted furyl, a substituted thienyl, a substituted pyrrolyl, a substituted imidazolyl, a substituted pyrazolyl, a substituted oxazolyl, a substituted thiazolyl, a substituted pyridinyl, a substituted pyranyl, a substituted pyrimidinyl, and a substituted pyrazinyl. The substituted fused-ring heteroaryl is selected from the group consisting of a substituted benzofuryl, a substituted benzothienyl, a substituted benzopyrrolyl, a substituted benzimidazolyl, a substituted benzoxazolyl, a substituted benzopyrazolyl, a substituted benzothiazolyl, a substituted benzopyranyl, a substituted quinolone, and a substituted acridine. A substituent in the substituted heteroaryl group is selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

In some embodiments, in the formula I, Ar₁ is a substituted phenyl, and Ar₂ is a substituted phenyl. Specifically, Ar₂ is a halogen atom substituted (C₁-C₁₀) alkyl substituted phenyl, for example, a halogenated C₁-C₁₀ alkyl monosubstituted or polysubstituted phenyl, and the like. The halogen atom can be fluorine, chlorine, bromine, Iodine. Ar₁ is (a (C₁-C₁₀) alkyl substituted phenyl, such as a methyl monosubstituted or polysubstituted phenyl, an ethyl monosubstituted or polysubstituted phenyl, and the like.

In an embodiment, Ar₁ is mesitylene, Ar₂ is 3,5-bis(trifluoromethyl)phenyl, and X is a sulfur atom or an oxygen atom. The structure of the nitrogen heterocyclic carbene catalyst is as follows:

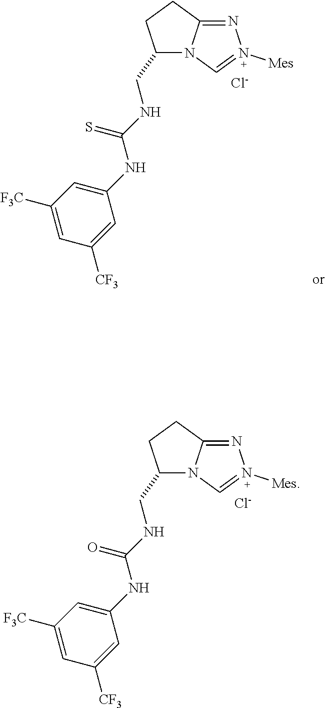

The selection of mesitylene as Ar₁ can improve an 6 electron donor ability of N-heterocyclic carbine to increase the Bronsted basicity of N-heterocyclic carbine, while the selection of 3,5-bis(trifluoromethyl)phenyl as Ar₂ can greatly improve the acidity of thiourea, which makes thiourea to be much easier to form hydrogen bonds with the substrate, thereby playing the catalytic activity and stereoselectivity. In this way, through the combined effects of these two unique groups, a general catalytic mode having better catalytic effect can be formed.

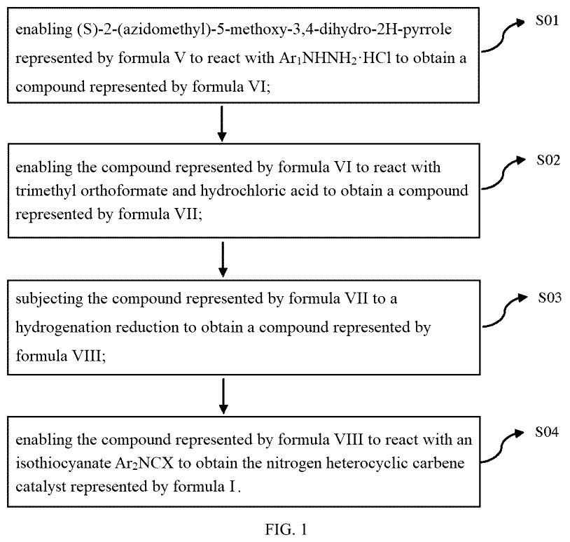

In another aspect, the present application also provides a method for preparing a nitrogen heterocyclic carbene catalyst. As shown in FIG. 1, the method comprises steps of:

S01: enabling (S)-2-(azidomethyl)-5-methoxy-3,4-dihy dro-2H-pyrrol e represented by formula V to react with Ar₁NHNH₂·HCl to obtain a compound represented by formula VI;

S02: enabling the compound represented by formula VI to react with trimethyl orthoformate and hydrochloric acid to obtain a compound represented by formula VII;

S03: subjecting the compound represented by formula VII to a hydrogenation reduction to obtain a compound represented by formula VIII;

S04: enabling the compound represented by formula VIII to react with an isothiocyanate Ar₂NCX to obtain the nitrogen heterocyclic carbene catalyst represented by formula I;

In which, the structural formulas of above-mentioned compound is as follows:

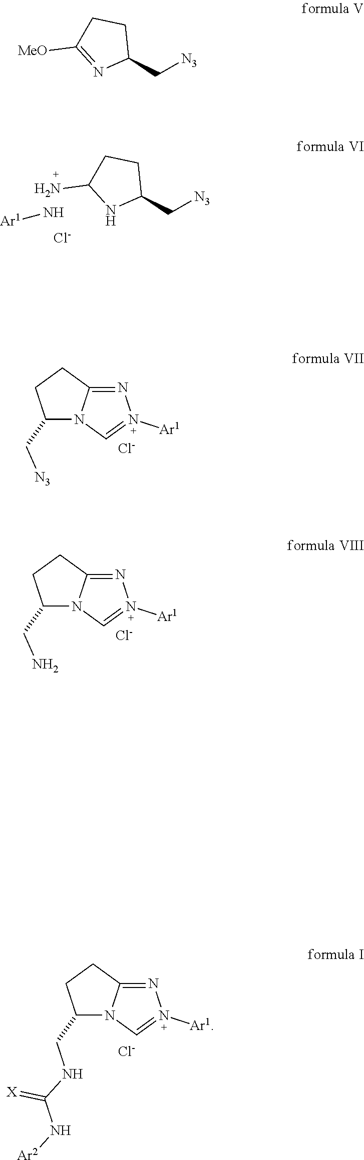

Ar₁ and Ar₂ in the formula I represent any one independently selected from the group consisting of an aryl, a substituted aryl, a heteroaryl, and a substituted heteroaryl, respectively; and X represents an oxygen atom or a sulfur atom.

Advantages of the method for preparing the nitrogen heterocyclic carbene catalyst provided by embodiments of the present application are summarized as follows: the preparation method is advantageous in easily accessible raw materials, low cost, convenient operation, mild reaction conditions, and easy control, and can be prepared into the bifunctional thiourea catalyst, that is, the nitrogen heterocyclic carbene catalyst, which has good catalytic activity in catalyzing asymmetric aza-Michael reactions, thereby having a potential application in the field of organic asymmetric catalysis.

Ar₁ in Ar₁NHNH₂·HCl and Ar₂ in Ar₂NCX respectively correspond to Ar₁ and Ar₂ in the nitrogen heterocyclic carbene catalyst represented by formula I, and the specific selection of Ar₁ and Ar₂ has been described in detail in the above.

In step S01, (S)-2-(azidomethyl)-5-methoxy-3,4-dihydro-2H-pyrrole represented by formula V is prepared by the following steps:

performing hydroxyl protection on L-pyroglutaminol represented by formula II using p-toluenesulfonyl chloride to obtain (S)-5-hydroxymethyl-2-pyrrolidone p-toluenesulfonate represented by formula III;

enabling (S)-5-hydroxymethyl-2-pyrrolidone p-toluenesulfonate represented by formula III to react with sodium azide to obtain (S)-5-azidomethyl-2-pyrrolidone represented by formula IV; and

enabling (S)-5-azidomethyl-2-pyrrolidone represented by formula IV to react with trimethyloxonium tetrafluoroborate to obtain (S)-2-(azidomethyl)-5-methoxy-3,4-dihydro-2H-pyrrole represented by formula V.

The structural formulas of above-mentioned compounds are as follows:

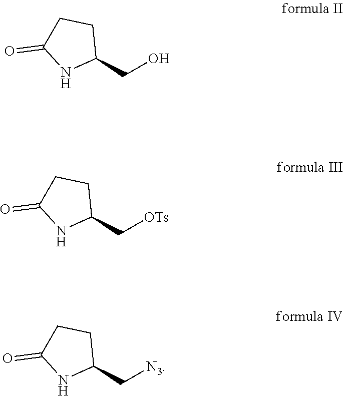 [SMILES: O=C1CC[C@@H](CO)N1 ; Cc1ccc(S(=O)(=O)OC[C@@H]2CCC(=O)N2)cc1 ; [N-]=[N+]=NC[C@@H]1CCC(=O)N1]

The above steps use L-pyroglutaminol as the raw material to synthesize the bifunctional nitrogen heterocyclic carbene catalyst. L-pyroglutaminol is a simple and easily accessible chemical raw material. The preparation method is advantageous in easily accessible raw materials, low cost, convenient operation, and has potential application in the field of organic asymmetric catalysis.

Specifically, L-pyroglutaminol is used as the raw material for synthesizing nitrogen heterocycle carbene catalyst, the synthetic route is as follows:

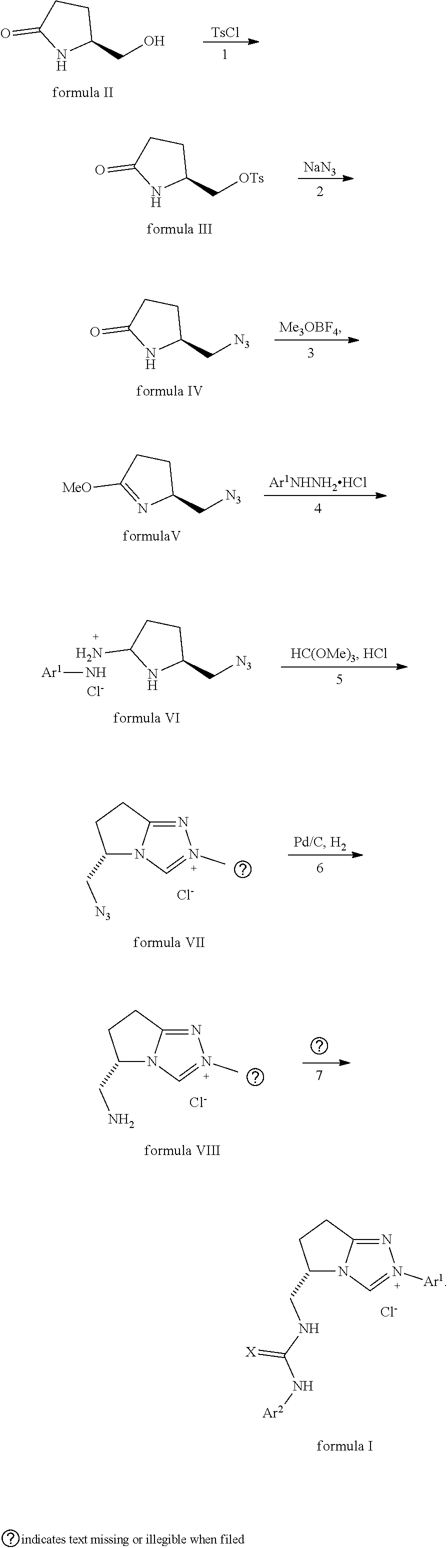

The synthetic route includes 7 steps, and a final product is obtained with a yield of 11%. The synthetic route is advantageous in easily accessible raw material, low production cost, convenient operation, mild reaction conditions, and easy control. Specifically, for example, Ar₁ is mesitylene, that is, Ar₁NHNH₂·HCl is mesitylenehydrazine hydrochloride, Ar₂ is 3,5-bis(trifluoromethyl)phenyl, X is a sulfur atom, that is, Ar₂NCX being 3,5-bis(trifluoromethyl)phenyl isothiocyanate, the synthetic method comprises the following steps:

In step 1, L-pyroglutaminol is used as a raw material, hydroxyl protection is performed on L-pyroglutaminol using p-toluenesulfonyl chloride (reactants include: p-toluenesulfonyl chloride, triethylamine, 4-lutidine, dichloromethane solution), to obtain an intermediate (S)-5-hydroxymethyl-2-pyrrolidone p-toluenesulfonate.

In step 2, the intermediate (S)-5-hydroxymethyl-2-pyrrolidone p-toluenesulfonate is enabled to react with sodium azide to obtain an intermediate (S)-5-azidomethyl-2-pyrrolidone.

In step 3, the intermediate (S)-5-azidomethyl-2-pyrrolidone is enabled to react with trimethyloxymonium tetrafluoroborate, to obtain an intermediate (S)-2-(azidomethyl)-5-methoxy-3,4-dihydro-2H-pyrrole.

In step 4, the intermediate (S)-2-(azidomethyl)-5-methoxy-3,4-dihydro-2H-pyrrole is enabled to react with mesitylenehydrazine hydrochloride to obtain an intermediate 1-((5 S)-5-(azidomethyl)pyrrolidin-2-yl)-2-mesitylenehydrazine hydrochloride.

In step 5, the intermediate 1-((5S)-5-(azidomethyl)pyrrolidin-2-yl)-2-mesitylenehydrazine hydrochloride is enabled to react with trimethyl orthoformate and hydrochloric acid, to obtain an intermediate (S)-5-(azidomethyl)-2-methanesulfonyl-6,7-dihydro-5H-pyrrolo[2,1-c][1,2,4]triazole-2-ium chloride.

In step 6, the intermediate (S)-5-(azidomethyl)-2-methylsulfonyl-6,7-dihydro-5H-pyrrolo[2,1-c][1,2,4]triazol-2-ium chloride is reductively hydrogenated with palladium carbon to obtain an intermediate (S)-5-(aminomethyl)-2-tolyl-6,7-dihydro-5H-pyrrolo[2,1-c][1,2,4]triazol-2-ium chloride.

In step 7, the intermediate (S)-5-(aminomethyl)-2-tolyl-6,7-dihydro-5H-pyrrolo[2,1-c][1,2,4]triazol-2-ium chloride is enabled to react with 3,5-bis(trifluoromethyl)phenyl isothiocyanate, to obtain (S)-5-43-(3,5-bis(trifluoromethyl)phenyl)thioureido)methyl)-2-mesityl-6,7-dihydro-5H-pyrrolo[2,1-c][1,2,4]triazol-2-ium chloride as a final product. (S)-5-43-(3,5-bis(trifluoromethyl)phenyl)thioureido)methyl)-2-mesityl-6,7-dihydro-5H-pyrrolo[2,1-c][1,2,4]triazol-2-ium chloride has a structural formula as follows:

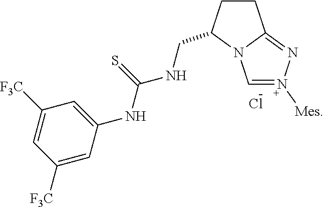

The nitrogen heterocyclic carbene catalyst provided by the present application and the nitrogen heterocyclic carbene catalyst prepared by the preparation method have good catalytic activity in catalyzing asymmetric aza-Michael reactions, for example: a nucleophile compound NR₁R₂ and a conjugated enone compound

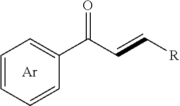

can be catalyzed by the nitrogen heterocyclic carbene catalyst of the present application in the asymmetric aza-Michael addition reaction, to obtain a β-carbonyl chiral amino compound. The reaction formula is as follows:

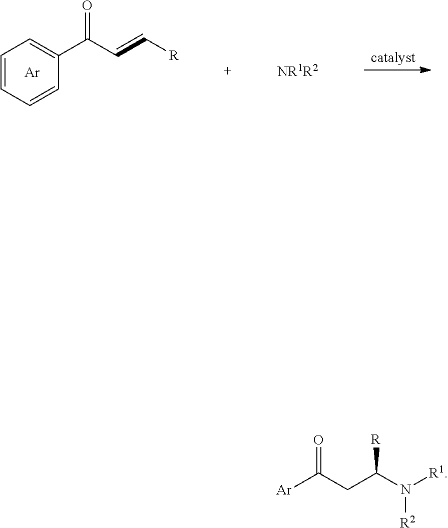

R₁ and R₂ in the nucleophile compound NR₁R₂ can be the same or different, and may be independently any one selected from the group consisting of a C₁-C₂₀ alkyl, a C₁-C₂₀ alkoxy, a C₁-C₂₀ alkyloxycarbonyl, an aryl, an aryloxy, an aryloxycarbonyl, an aryl(C₁-C₂₀)alkyl, an aryl(C₁-C₂₀)alkoxy, and an aryl(C₁-C₂₀)alkoxycarbonyl. R in the conjugated enone compounds can be a C₁-C₂₀ alkyl, a C₁-C₂₀ heteroalkyl, a C₃-C₂₀ cycloalkyl, a C₃-C₂₀ heterocycloalkyl, a C₂-C₂₀ alkenyl, a C₂-C₂₀ heteroalkenyl, a C₃-C₂₀ cycloalkenyl, a C₃-C₂₀ heterocycloalkenyl, a C₂-C₂₀ alkynyl, a C₂-C₂₀ heteroalkynyl, a C₃-C₂₀ cycloalkynyl, a C₃-C₂₀ heterocycloalkynyl, a C₁-C₂₀ alkoxy, a C₁-C₂₀ alkyloxycarbonyl, a C₁-C₂₀ alkyloxycarbonyl (C₁-C₂₀) alkyl, an aryl, a substituted aryl, a heteroaryl, a substituted heteroaryl, an aryloxy, an aryloxy carbonyl, a heteroaryloxy, a heteroaryloxycarbonyl, an aryl(C₁-C₂₀)alkyl, an aryl(C₁-C₂₀)alkoxy, a heteroaryl(C₁-C₂₀)alkyl, a C₂-C₂₀ alkenyl (C₁-C₂₀) alkyl, a C₂-C₂₀ alkynyl (C₁-C₂₀) alkyl, a cyano (C₁-C₂₀) alkyl, and a halo (C₁-C₂₀) alkyl. Various kinds of β-carbonyl chiral amino compounds can be obtained through the catalytic action of the nitrogen heterocyclic carbene catalyst of the present application.

The present application has been tested many times successively, and some test results are given as reference to further describe the present application in detail, and the following will be described in detail in conjunction with specific examples.

### Example 1

A bifunctional nitrogen heterocyclic carbene catalyst has a molecular structure as follows:

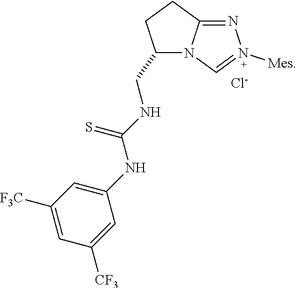

A method for preparing the bifunctional nitrogen heterocyclic carbene catalyst comprised the following steps:

### Step (1): Synthesis of an intermediate (S)-5-hydroxymethyl-2-pyrrolidone p-toluenesulfonate, in which, the reaction formula and synthesis steps were as follows

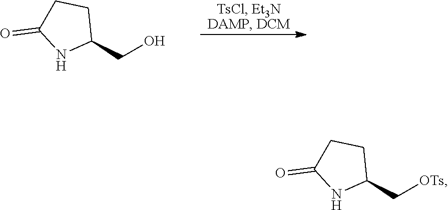

10 g of L-pyroglutaminol and 21 g of p-toluenesulfonyl chloride were added to 300 mL of a dichloromethane solution to yield a mixture. The resulting mixture was cooled to a temperature of 0° C., then added with 2.2 g of 4-lutidine and 16 mL of triethylamine. A resulting mixture was then returned to the room temperature, stirred for 12 hrs and a sample was taken for monitoring a progress of the reaction (.by a thin-layer chromatography detection, TLC detection). After the reaction was complete, a resulting reaction system was quenched with water and extracted with a dichloromethane solution, thereafter, organic phases were combined and dried with an anhydrous sodium sulfate, and then a product was obtained by recrystallization. 21.7 g of solid product was obtained, and a yield was 93%.

### Step (2): Synthesis of intermediate (S)-5-azidomethyl-2-pyrrolidone, in which, the reaction formula and synthesis steps were as follows

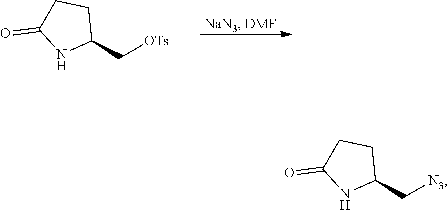

15 g of (S)-5-hydroxymethyl-2-pyrrolidone p-toluenesulfonate was dissolved in 200 mL of a N,N-dimethylformamide solution, 4 g of a sodium azide solid was carefully added using a plastic spoon, and a reaction system was heated for 9 hrs at a temperature of 55° C., during which, the progress of the reaction (TLC detection) was monitored. After that, a resulting reaction system was filtered, and a filter residue was washed using ethyl acetate. A filtrate was rotary evaporated and purified by a column chromatography to obtain a light yellow oily liquid. 7.0 g of a liquid product was obtained, and a yield was 90%.

### Step (3): Synthesis of intermediate (S)-2-(azidomethyl)-5-methoxy-3,4-dihydro-2H-pyrrole, in which, the reaction formula and synthesis steps were as follows

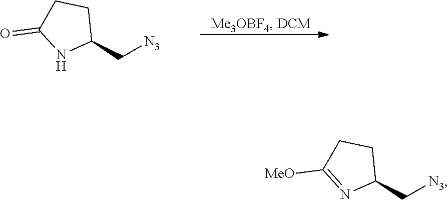

6.0 g of (S)-5-azidomethyl-2-pyrrolidone and 7.0 g of trimethyloxyazine tetrafluoroborate were dissolved in 300 mL of dichloromethane solution. A resulting reaction system was stirred at room temperature for 17 hrs under an atmosphere of nitrogen protection, during which, a progress of the reaction was monitored. The reaction system was cooled to 0° C., quenched with a saturated sodium bicarbonate solution, and then continued to be stirred for 1 hr, and then extracted. An organic phase was spin-dried, and an obtained product was directly fed into a next step.

### Step (4): Synthesis of an intermediate 1-((5S)-5-(azidomethyl)pyrrolidin-2-yl)-2-mesitylenehydrazine hydrochloride, in which, a reaction formula and synthesis steps were as follows

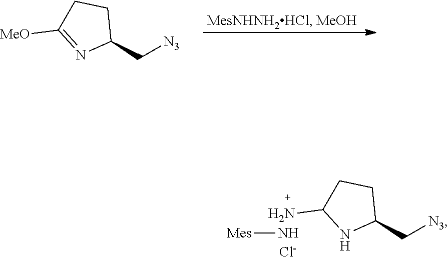

5.5 g of (S)-2-(azidomethyl)-5-methoxy-3,4-dihydro-2H-pyrrole was placed in a reaction flask, 6.6 g of mesitylenehydrazine hydrochloride and 140 mL of methanol solution were subsequently added, a resulting reaction system was stirred at 50° C. for 1 hr, and samples were taken to monitor the process of reaction (TLC detection). After that, the reaction was cooled to room temperature and spin-dried to obtain a solid. The solid was then dissolved in ethyl acetate and filtered off with suction. A filtrate was concentrated and spin-dried, and a resulting product was fed directly to a next step.

### Step (5): synthesis of an intermediate V, (S)-5-(azidomethyl)-2-methylsulfonyl-6,7-dihydro-5H-pyrrolo[2,1-c][1,2,4]triazol-2-ium chloride, in which, the reaction formula and synthetic steps were as follows

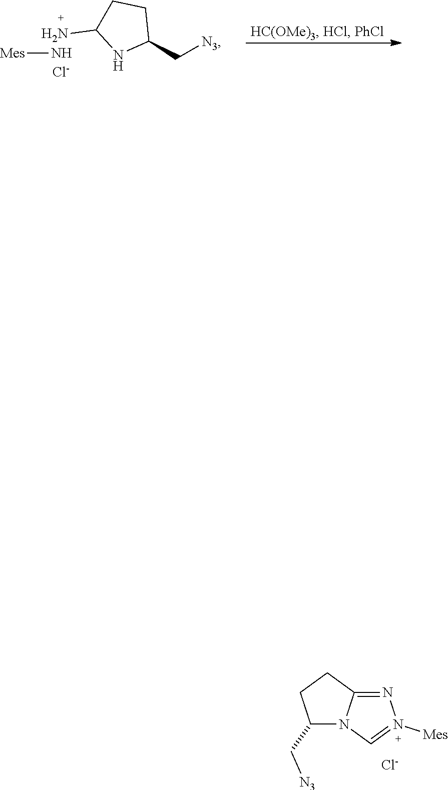

The solid obtained in the previous step was placed in a reaction flask, 38.0 mL of trimethyl orthoformate, 28.0 mL of a chlorobenzene solution and 7.0 mL of a concentrated hydrochloric acid were added subsequently, and nitrogen was introduced to a resulting mixture for replacing an original gas. After that, the resulting mixture was refluxed at 100° C. for 1 hr, and the reaction system was monitored (TLC detection). After the reaction was complete, the reaction system was returned to room temperature, spin-dried, and purified by a column chromatography to obtain a brown solid, and purified by a column chromatography to obtain a brown solid. The obtained brown solid was 4.45 g, and a yield thereof was 50%.

### Step (6): Synthesis of an intermediate (S)-5-(aminomethyl)-2-tolyl-6,7-dihydro-5H-pyrrolo[2,1-c][1,2,4]triazole-2-ium chloride, in which, the synthetic steps were as follows

1.5 g of (S)-5-(aminomethyl)-2-tolyl-6,7-dihydro-5H-pyrrolo[2,1-c][1,2,4]triazole-2-ium chloride was placed in a reaction flask, 148 mg of a 10% palladium carbon was added, and hydrogen was used to reduce for 12 hours, and samples were taken for monitoring the progress of the reaction (TLC detection). After filtering off the palladium carbon, the filtrate was spin-dried and purified by a column chromatography to obtain a tan solid. The obtained tan solid was1.34 g, and a yield thereof was 91%.

### Step (7): Synthesis of a final product (S)-5-43-(3,5-bis(trifluoromethyl)phenyl)thioureido)methyl)-2-mesityl-6,7-dihydro-5H-pyrrolo[2,1-c][1,2,4]triazol-2-ium chloride, the reaction formula and synthesis steps were as follows

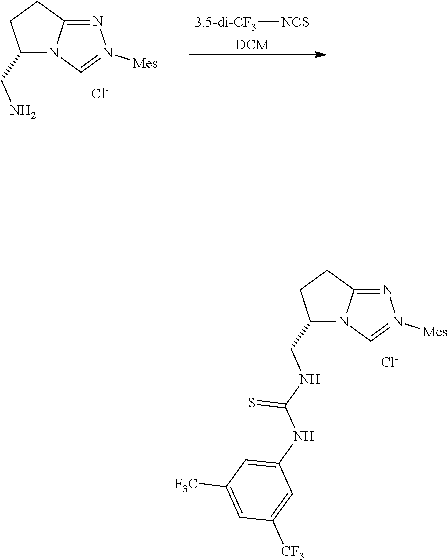

250 mg of a solid obtained in the previous step was placed into a reaction flask, 230 mg of 3,5-bis(trifluoromethyl)phenyl isothiocyanate and 2.5 mL of a dichloromethane solution were added to the reaction flask, a resulting mixture was stirred at room temperature for 12 hrs, during which, samples was taken to monitor a progress of the reaction (TLC detection). After the reaction was complete, the reaction system was spin-dried and purified by a column chromatography, and a white solid was obtained. The obtained solid product was 440 mg, and the yield was 95%.

Correlation characterization analyses were performed and results were as follows: 1 HNMR (400 MHz, CDCl₃) δ 10.91 (s, 1H), 10.77 (s, 1H), 9.45 (t, J=6.0 Hz, 1H), 8.23 (d, J=1.6 Hz, 2H), 7.53 (s, 1H), 6.89 (s, 2H), 5.32 (dq, J=10.2, 6.7, 5.3 Hz, 1H), 4.27 (tt, J=14.5, 8.1 Hz, 2H), 3.43 (ddd, J=17.5, 9.3, 5.8 Hz, 1H), 3.35-3.16 (m, 1H), 3.05 (dtd, J=14.2, 8.5, 6.0 Hz, 1H), 2.84-2.59 (m, 1H), 2.28 (s, 3H), 2.05 (s, 6H). ¹³C NMR (101 MHz, Chloroform-d) δ 182.52, 161.72, 142.21, 141.66, 141.09, 134.73, 131.60, 131.31 (q, J=33.5 Hz), 129.78, 123.22 (q, J=272.8 Hz), 122.50, 117.35, 61.09, 45.43, 30.98, 21.69, 21.12, 17.74. 19F NMR (376 MHz, CDCl₃) δ-62.85. HRMS (ESI-TOF) [M⁺] calculated for [C₂₄H₂₄F₆N₅S]⁺528.1651, observed 528.1645. [α]_{D} 25=7.3 (c=0.80 in CHCl₃)

### Example 2

A bifunctional nitrogen heterocyclic carbene catalyst has a molecular structure as follows:

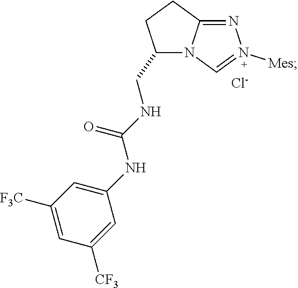

A method for preparing the bifunctional nitrogen heterocyclic carbene catalyst comprised the following steps:

The step (1)-step (6) were the same as those in Example 1;

Step (7) synthesis of a final product (S)-5-43-(3,5-bi s (trifluoromethyl)phenyl)ureido)methyl)-2-mesityl-6,7-dihy dro-5H-pyrrolo[2,1-c][1,2,4]triazol-2-ium chloride, in which, the reaction formula and synthesis steps were as follows:

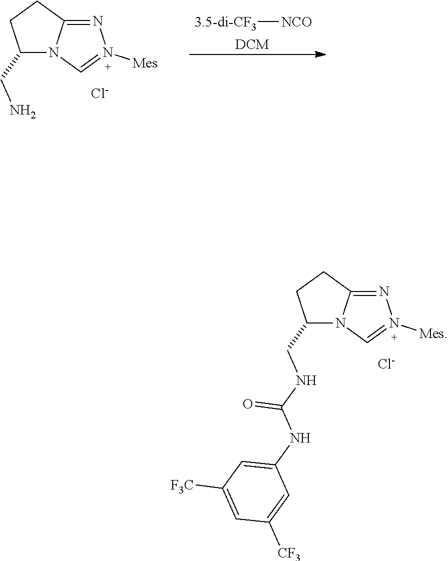

250 mg of a solid obtained in the previous step was placed into a reaction flask, 230 mg of 3,5-bis(trifluoromethyl)phenyl isocyanate and 2.5 mL of a dichloromethane solution were added to the reaction flask, a resulting mixture was stirred at room temperature for 12 hrs, during which, samples was taken to monitor a progress of the reaction (TLC detection). After the reaction was complete, the reaction system was spin-dried and purified by a column chromatography, and a white solid was obtained. The obtained solid product was 448 mg, and the yield was 93%.

Correlation characterization analyses were performed and results were as follows: ¹HNMR (400 MHz, Chloroform-d) δ 10.78 (s, 1H), 9.67 (s, 1H), 7.98 (d, J=6.3 Hz, 1H), 7.92 (s, 2H), 7.39 (s, 1H), 6.88 (s, 2H), 5.21-5.09 (m, 1H), 3.93 (dt, J=14.8, 4.5 Hz, 1H), 3.74 (dt, J=15.1, 7.6 Hz, 1H), 3.47-3.33 (m, 1H), 3.23 (ddd, J=17.5, 9.4, 5.6 Hz, 1H), 3.03 (ddd, J=16.9, 14.2, 8.0 Hz, 1H), 2.71 (ddq, J=14.6, 9.3, Hz, 1H), 2.25 (s, 3H), 2.03 (s, 6H). ¹³C NMR (101 MHz, Chloroform-d) δ 161.66, 155.95, 142.18, 141.81, 141.37, 134.76, 131.70 (q, J=33.2 Hz), 131.62, 129.68, 123.35 (q, J=272.6 Hz), 117.81, 114.79, 61.95, 41.84, 30.60, 21.74, 21.06, 17.50. 19F NMR (376 MHz, Chloroform-d) δ-62.96. HRMS (ESI-TOF) [M+H] calculated for [C₂₄H₂₄F₆N₅O]⁺512.1880, observed 512.1880. [α]²⁵_{D}=8.5 (c=0.80 in CHCl₃).

### Example 3

The asymmetric aza-Michael reaction was catalyzed by the bifunctional nitrogen heterocyclic carbene catalyst of Example 1, and the reaction formula and steps were as follows:

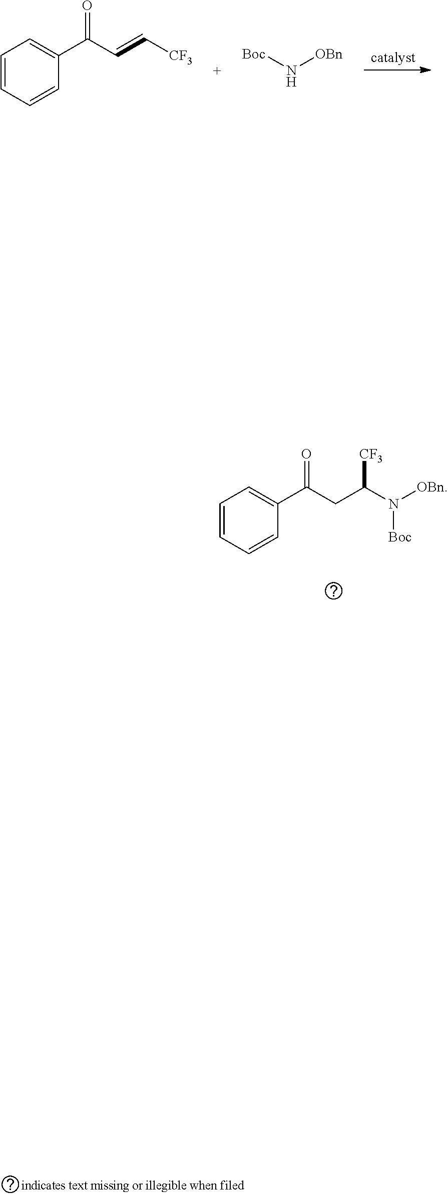

To a dried 10 mL test tube, a bifunctional nitrogen heterocyclic carbene catalyst (0.02 mmol, 0.2 equivalent) of Example 1 and 0.6 mL of anhydrous toluene were added. A resulting mixture was replaced with argon three times. An alkali (0.02 mmol, 0.2 equivalent)) was added, the test tube was then sealed, and a resulting mixture was stirred at a room temperature for 30 mins. The nucleophile tert-butyl carbamate (0.12 mmol, 1.2 equivalent) was slowly added to the reaction system and stirred at room temperature for 0.5 hr. A corresponding β-trifluoromethylketene (0.1 mmol, 1.0 equivalent) was slowly added to the reaction system, and the resulting mixture was stirred at room temperature for 12 hrs. After the reaction was completed, the reaction solution was filtered through a glass dropper containing a silica gel, rinsed with ether, a filtrate was spin-dried and separated by a column chromatography, and a colorless oily liquid was obtained as the target product.

Correlation characterization analyses were performed and results were as follows: ¹H NMR (400 MHz, Chloroform-d) δ 7.93-7.84 (m, 2H), 7.56 (t, J=7.4 Hz, 1H), 7.42 (t, J=7.7 Hz, 2H), 7.37-7.27 (m, 5H), 5.48-5.32 (m, 1H), 4.89 (dd, J=27.1, 9.7 Hz, 2H), 3.74 (dd, J=17.7, 9.7 Hz, 1H), 3.17 (dd, J=17.7, 3.4 Hz, 1H), 1.57 (s, 9H). ¹³C NMR (101 MHz, CDCl 3) δ 194.57, 156.17, 136.15, 135.15, 133.62, 129.40, 128.74, 128.65, 128.53, 128.28, 125.24 (q, J=282.1 Hz), 83.07, 78.15, 56.47 (q, J=31.1 Hz), 33.68, 28.26. 19F NMR (376 MHz, CDCl₃) δ-73.22. HRMS (ESI-TOF) [M+Na] calculated for [C₂₂H₂₄F₃NO₄Na]⁺446.1550, observed 446.1547. HPLC (Chiralpak-OJ column, 98: 2 hexane/ethanol, flow rate: 1.0 mL/min): tₘₐⱼₒᵣ=10.433 min; tₘᵢₙₒᵣ=7.081 min. [α]_{D}²⁵=−19.9 (c=0.80 in CHCl₃).

### Example 4

The bifunctional nitrogen heterocyclic carbene catalyst of Example 2 was used to catalyze an asymmetric aza-Michael reaction, and the reaction formula and steps were as follows:

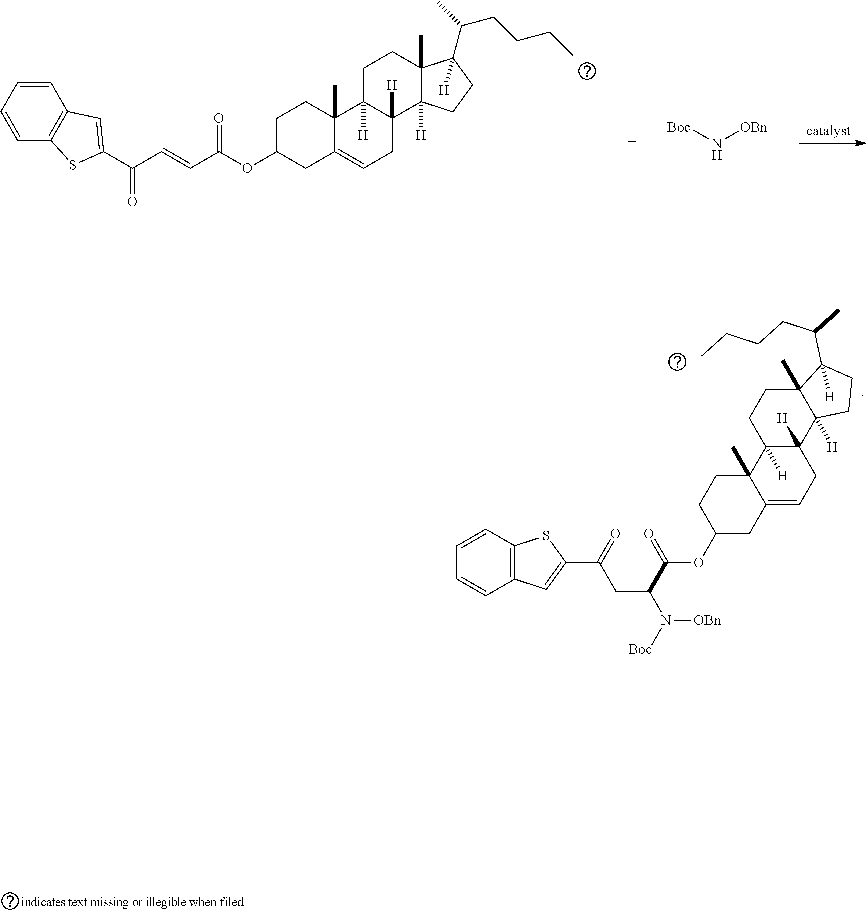

To a dried 10 mL test tube, a bifunctional nitrogen heterocyclic carbene catalyst (0.02 mmol, 0.2 equivalent) of Example 2 and 0.6 mL of anhydrous toluene were added. A resulting mixture was replaced with argon three times. An alkali (0.02 mmol, 0.2 equivalent)) was added, the test tube was then sealed, and a resulting mixture was stirred at a room temperature for 30 mins. The nucleophile tert-butyl carbamate (0.12 mmol, 1.2 equivalent) was slowly added to the reaction system and stirred at room temperature for 0.5 hr. A corresponding β-trifluoromethylketene (0.1 mmol, 1.0 equivalent) was slowly added to the reaction system, and the resulting mixture was stirred at room temperature for 12 hrs. After the reaction was completed, the reaction solution was filtered through a glass dropper containing a silica gel, rinsed with ether, a filtrate was spin-dried and separated by a column chromatography, and a colorless oily liquid was obtained as the target product, with a yield of 87% and 92% ee.

Correlation characterization analyses were performed and results were as follows: ¹HNMR (400 MHz, Chloroform-d) δ 7.86 (d, J=7.7 Hz, 3H), 7.48-7.35 (m, 4H), 7.35-7.28 (m, 3H), 5.34 (s, 1H), 5.26-5.21 (m, 1H), 4.91 (dd, J=47.0, Hz, 2H), 4.71-4.63 (m, 1H), 3.71 (ddd, J=16.8, 7.3, 2.7 Hz, 1H), 3.18 (dd, J=16.4, 4.8 Hz, 1H), 2.40-2.24 (m, 2H), 2.03-1.91 (m, 2H), 1.89-1.78 (m, 3H), 1.59 (d, J=34.8 Hz, 5H), 1.53 (s, 9H), 1.49-1.39 (m, 4H), 1.37-1.22 (m, 5H), 1.18-1.04 (m, 7H), 0.95 (s, 3H), 0.91 (d, J=6.5 Hz, 3H), 0.87 (d, J=1.8 Hz, 3H), 0.85 (d, J=1.8 Hz, 3H), 0.66 (s, 3H). ¹³C NMR (101 MHz, Chloroform-d) δ 191.08 (d, J=2.6 Hz), 168.95 (d, J=2.8 Hz), 156.74, 143.15, 142.63, 139.41, 139.05, 135.73, 129.59 (d, J=3.8 Hz), 128.48, 128.41, 127.50, 126.00, 125.02, 122.98, 122.83 (d, J=2.4 Hz), 82.43, 77.68 (d, J=2.0 Hz), 75.77, 59.69 (d, J=4.6 Hz), 56.67, 56.14, 49.98, 42.31, 39.72, 39.52, 38.56 (d, J=3.7 Hz), 37.96, 37.88, 36.90 (d, J=2.0 Hz), 36.55, 36.19, 35.79, 31.86 (d, J=5.1 Hz), 28.26, 28.23, 28.09, 28.02, 27.68, 27.59, 24.28, 23.84, 22.83, 22.57, 21.02, 19.24, 18.72, 11.85. HRMS (ESI-TOF) [M+Na] calculated for [C₅₁H₆₉NO₆SNa]⁺846.4738 observed 846.4740. HPLC (Chiralpak-IA column, 97.5: 2.5 hexane/ethanol, flow rate: 1.0 mL/min): tₘₐⱼₒᵣ=11.946 min; tₘᵢₙₒᵣ=min. [α]_{D}²⁵=−21.1 (c=0.80 in CHCl₃).

The aforementioned embodiments are only preferred embodiments of the present application, and are not intended to limit the present application. Any modification, equivalent replacement, improvement, and so on, which are made within the spirit and the principle of the present application, should be included in the protection scope of the present application.

1. A nitrogen heterocyclic carbene catalyst, having a structural formula represented by formula I: 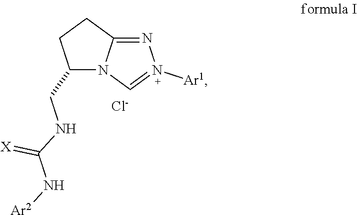
wherein
Ar¹ and Ar² in the formula I represent any one independently selected from the group consisting of an aryl, a substituted aryl, a heteroaryl, and a substituted heteroaryl, respectively; and X represents an oxygen atom or a sulfur atom.

2. The nitrogen heterocyclic carbene catalyst according to claim 1, wherein the aryl group is at least one selected from the group consisting of phenyl, naphthyl, anthracenyl, phenanthrenyl, and fluorenyl.

3. The nitrogen heterocyclic carbene catalyst according to claim 1, wherein the heteroaryl is at least one selected from the group consisting of a monocyclic heteroaryl and a fused-ring heteroaryl.

4. The nitrogen heterocyclic carbene catalyst according to claim 3, wherein the monocyclic heteroaryl is at least one selected from the group consisting of furyl, thienyl, pyrrolyl, imidazolyl, pyrazolyl, oxazolyl, thiazolyl, pyridinyl, pyranyl, pyrimidinyl, and pyrazinyl; and/or
the fused-ring heteroaryl is at least one selected from the group consisting of benzofuryl, benzothienyl, benzopyrrolyl, benzimidazolyl, benzoxazolyl, benzopyrazolyl, benzothiazolyl, benzopyranyl, quinolone, and acridine.

5. The nitrogen heterocyclic carbene catalyst according to claim 1, wherein the substituted aryl is at least one selected from the group consisting of a substituted phenyl, a substituted naphthyl, a substituted anthracenyl, a substituted phenanthrenyl, and a substituted fluorenyl.

6. The nitrogen heterocyclic carbene catalyst according to claim 5, wherein a substituent in the substituted aryl is at least one selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

7. The nitrogen heterocyclic carbene catalyst according to claim 1, wherein the substituted heteroaryl is selected from at least one of a substituted monocyclic heteroaryl and a substituted fused-ring heteroaryl.

8. The nitrogen heterocyclic carbene catalyst according to claim 7, wherein
a substituted monocyclic heteroaryl is selected from the group consisting of a substituted furyl, a substituted thienyl, a substituted pyrrolyl, a substituted imidazolyl, a substituted pyrazolyl, a substituted oxazolyl, a substituted thiazolyl, a substituted pyridinyl, a substituted pyranyl, a substituted pyrimidinyl, and a substituted pyrazinyl; and/or
the substituted fused-ring heteroaryl is selected from the group consisting of a substituted benzofuryl, a substituted benzothienyl, a substituted benzopyrrolyl, a substituted benzimidazolyl, a substituted benzoxazolyl, a substituted benzopyrazolyl,a substituted benzothiazolyl, a substituted benzopyranyl, a substituted quinolone, and a substituted acridine.

9. The nitrogen heterocyclic carbene catalyst according to claim 7, wherein a substituent in the substituted heteroaryl group is selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

10. The nitrogen heterocyclic carbene catalyst according to claim 1, wherein in the formula I, Ar¹ is a substituted phenyl, and Ar² is a substituted phenyl.

11. The nitrogen heterocyclic carbene catalyst according to claim 10, wherein in the formula I, Ar¹ is a (C₁-C₁₀) alkyl substituted phenyl, and Ar² is a phenyl substituted by a halogen substituted (C₁-C₁₀) alkyl.

12. The nitrogen heterocyclic carbene catalyst according to claim 11, wherein in the formula I, Ar¹ is mesitylene, and Ar² is 3,5-bis(trifluoromethyl)phenyl.

13. A method for preparing a nitrogen heterocyclic carbene catalyst, comprising steps of:
enabling (S)-2-(azidomethyl)-5-methoxy-3,4-dihydro-2H-pyrrole represented by formula V to react with Ar¹NHNH₂·HCl to obtain a compound represented by formula VI;
enabling the compound represented by formula VI to react with trimethyl orthoformate and hydrochloric acid to obtain a compound represented by formula VII;
subjecting the compound represented by formula VII to a hydrogenation reduction to obtain a compound represented by formula VIII; and
enabling the compound represented by formula VIII to react with an isothiocyanate Ar²NCX to obtain the nitrogen heterocyclic carbene catalyst represented by formula I; wherein structural formulas of formula V, formula VI, formula VII, formula VIII, formula I are as follows: 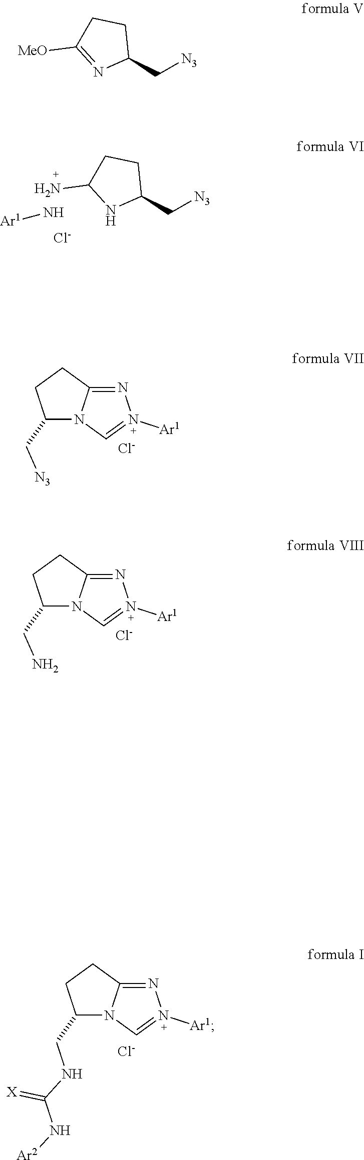
and
Ar¹ and Ar² in the formula I represent any one independently selected from the group consisting of an aryl, a substituted aryl, a heteroaryl, and a substituted heteroaryl, respectively; and X represents an oxygen atom or a sulfur atom.

14. The nitrogen heterocyclic carbene catalyst according to claim 13, wherein (S)-2-(azidomethyl)-5-methoxy-3,4-dihydro-2H-pyrrole represented by formula V is prepared by the following steps:
performing hydroxyl protection on L-pyroglutaminol represented by formula II using p-toluenesulfonyl chloride to obtain (S)-5-hydroxymethyl-2-pyrrolidone p-toluenesulfonate represented by formula III;
enabling (S)-5-hydroxymethyl-2-pyrrolidone p-toluenesulfonate represented by formula III to react with sodium azide to obtain (S)-5-azidomethyl-2-pyrrolidone represented by formula IV; and
enabling (S)-5-azidomethyl-2-pyrrolidone represented by formula IV to react with trimethyloxonium tetrafluoroborate to obtain (S)-2-(azidomethyl)-5-methoxy-3,4-dihydro-2H-pyrrole represented by formula V;
wherein, the structural formulas of formula II, formula III, formula IV are as follows:

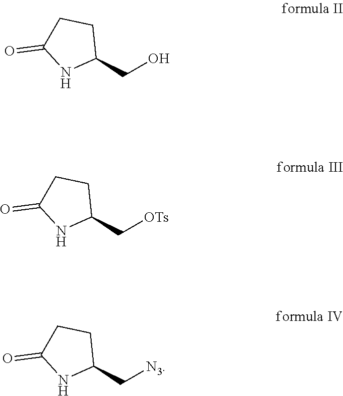 [SMILES: O=C1CC[C@@H](CO)N1 ; Cc1ccc(S(=O)(=O)OC[C@@H]2CCC(=O)N2)cc1 ; [N-]=[N+]=NC[C@@H]1CCC(=O)N1]

15. The nitrogen heterocyclic carbene catalyst according to claim 13, wherein
the aryl group is at least one selected from the group consisting of phenyl, naphthyl, anthracenyl, phenanthrenyl, and fluorenyl;
the heteroaryl is at least one selected from the group consisting of a monocyclic heteroaryl and a fused-ring heteroaryl;
the substituted aryl is at least one selected from the group consisting of a substituted phenyl, a substituted naphthyl, a substituted anthracenyl, a substituted phenanthrenyl, and a substituted fluorenyl; and a substituent in the substituted aryl is at least one selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl; and
the substituted heteroaryl is selected from at least one of a substituted monocyclic heteroaryl and a substituted fused-ring heteroaryl; and a substituent in the substituted heteroaryl group is selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

16. The nitrogen heterocyclic carbene catalyst according to claim 13, wherein the aryl group is at least one selected from the group consisting of phenyl, naphthyl, anthracenyl, phenanthrenyl, and fluorenyl.

17. The nitrogen heterocyclic carbene catalyst according to claim 13, wherein the heteroaryl is at least one selected from the group consisting of a monocyclic heteroaryl and a fused-ring heteroaryl;

18. The nitrogen heterocyclic carbene catalyst according to claim 13, wherein the substituted aryl is at least one selected from the group consisting of a substituted phenyl, a substituted naphthyl, a substituted anthracenyl, a substituted phenanthrenyl, and a substituted fluorenyl.

19. The nitrogen heterocyclic carbene catalyst according to claim 18, wherein a substituent in the substituted aryl is at least one selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.

20. The nitrogen heterocyclic carbene catalyst according to claim 13, wherein the substituted heteroaryl is selected from at least one of a substituted monocyclic heteroaryl and a substituted fused-ring heteroaryl; and a substituent in the substituted heteroaryl group is selected from the group consisting of a halogen atom, a hydroxyl, an amino, a nitro, a sulfo, a cyano, an acyl, an ester, a (C₁-C₁₀) alkyl, a (C₆-C₁₄) aryl, and a (C₄-C₁₄) heteroaryl.
